(19) United States
(12) Patent Application Publication
(10) Pub. No.: US 2022/0118131 A1
(43) Pub. Date: Apr. 21, 2022

(54) DEVICE TO PREVENT CATHETER ASSOCIATED URINARY TRACT INFECTION

(71) Applicant: SECRETARY, DEPARTMENT OF BIOTECHNOLOGY, New Delhi (IN)

(72) Inventors: Nirmal KUMAR, Distt-bharaich (IN); Aniket Anand KULKARNI, Pune (IN); Deepika DIXIT, Pune (IN); Yasuyuki MATSUURA, Higashi-Hiroshima City (JP); Prashant JHA, New Delhi (IN); Harpal SINGH, New Delhi (IN); Hitender GAUTAM, New Delhi (IN)

(21) Appl. No.: 17/299,446

(22) PCT Filed: Dec. 3, 2019
(86) PCT No.: PCT/IN2019/050878

§ 371 Date: Jun. 3, 2021

(30) Foreign Application Priority Data
Dec. 3, 2018 (IN) 201811045665

(51) Int. Cl. A61L 2/10; A61L 2/08; A61L 2/26

(52) U.S. Cl. CPC A61L 2/10; A61L 2/088; A61L 2202/11; A61L 2202/24; A61L 2/26

(57) ABSTRACT

The present invention discloses the device for a urinary catheter employing electromagnetic radiation and/or vibration transducer. The device comprises a clip-on (4) including a source of electromagnetic radiation (5) and/or a vibration transducer and a coupler (3) that allows electromagnetic radiation access from clip-on to the inside of the coupler. The combination of electromagnetic radiation and photo catalyst material may increase effectiveness for antimicrobial activity. The device aims at preventing the catheter-associated urinary tract infection caused by both intraluminal and extraluminal routes.

### FIELD OF THE INVENTION

The subject matter of the present invention, in general, pertains to the field of catheters, and more particularly, to a device to prevent catheter associated urinary tract infections.

### BACKGROUND OF INVENTION

A catheter is a thin tube made from medical grade materials to serve a broad range of functions. In particular, they are medical devices that can be inserted into a body to treat diseases or perform surgical procedures. Catheters are manufactured for cardiovascular, urological, gastrointestinal, neurovascular, and ophthalmic applications. The process of inserting a catheter is “catheterization”.

A urinary catheter is a hollow, partially flexible tube that collects urine from the bladder and leads to a drainage bag. One may need a catheter if one has had surgery or cannot control one's bladder function. Urinary catheters come in many sizes and types. Catheters are generally necessary when someone can't empty their bladder. If the bladder isn't emptied, urine can build up and lead to pressure in the kidneys. The pressure can lead to kidney failure, which can be dangerous and result in permanent damage to the kidneys. There are three main types of catheters: a) indwelling catheters, b) external catheters, and c) short-term catheters.

Indwelling catheters, also known as urethral or suprapubic catheters is a catheter that resides in the urethra for short and long periods of time. It drains urine into a collection bag. A tiny balloon at the end of the catheter is inflated with water to prevent the tube from sliding out of the body. The balloon can then deflate when the catheter needs to be removed.

A urinary tract infection (UTI) is an infection involving any part of the urinary system, including urethra, bladder, ureters, and kidney. The in-dwelling urinary catheters are used routinely during many surgical procedures and by patients suffering from urinary incontinence or by disabled individuals like paraplegics or tetraplegics, who may have no control over urination. Catheterisation may be the only available way of managing urination. Urinary catheters exist in combination with bags for collecting the urine which can be emptied into a toilet.

The most important risk factor for developing a catheter-associated UTI (CAUTI) is prolonged use of the urinary catheter. In fact, catheterisation itself can introduce microorganisms such as bacteria or fungi into the urinary tract and/or the bladder of a user. There is a huge risk of bacteria or fungi entering the urinary tract via the in-dwelling urinary catheter and multiply therein, causing an infection. There are a number of ways infection can occur during catheterization. For example: backflow of urine into the bladder, unsanitary insertion techniques, bacteria from bowel movement entering the catheter, size of catheter, etc. Therefore, catheters should only be used for appropriate indications and should be removed as soon as they are no longer needed.

Reference is made to publication number US 2015/0126976 A1, wherein a device enclosed with UV illuminator is used as a self-sterilizing device for the catheter and other medical devices is disclosed. The device comprises a tubular member which is flexible and configured to receive ultraviolet (UV) light from a UV illumination coupler. The tubular member contains a lumen defining a longitudinal interior space within the tubular member, a tubular body bounded by an inner wall defining an outer boundary of the lumen and an outer wall defining an outer surface of the tubular member, at least one optical fibre disposed outside of the interior space not parallel to an axis of the lumen and adapted to receive the UV light from the UV illumination coupler, and a protective component adapted to prevent substantively all of the UV light emitted from the optical fibre from exiting the outer wall.

In regard of the same, another reference is made to U.S. Pat. No. 9,550,005B2 that discloses a method for sterilizing a catheter with at least a first lumen, into which source of light is inserted and withdrawn to sterilize the catheter.

Reference is also made to US 2007/0244423 A1, wherein an acoustic clip-on device over the connecter of catheter and bag is disclosed. This device is associated with an external piezo-element transducer or vibrator and because of acoustic wave the biofilm in-side the catheter is dislodged. The clip-on device applies surface acoustic waves of Rayleigh-Lamb and/or Love type to a urinary catheter for preventing biofilms on the catheter surfaces. The bacteria are forced to move relative to the vibrating catheter surface.

A further reference is made to US 2005/0038376 A1, wherein a piezo-ceramic element based device is attached to the catheter is disclosed. The device when supplied with electricity the ceramic element generates vibration resulting in the displacement of biofilm. The vibration processor provides electric signals that generate acoustic vibrations in the piezo-ceramic element, causing vibrations in or around the catheter to disperse microbe colonies, thereby preventing or inhibiting formation of biofilm that may lead to infections.

Yet another reference is made to WO2003099100 that discloses an apparatus, system and method for preventing or treating biofilm associated with catheters. It discloses the that the apparatus comprises a processor to supply at least one electric signal to a piezo-ceramic element, and at least one electric signal causes the piezo-ceramic element to generate vibrations thereby treating the biofilm.

Further reference is drawn to U.S. Ser. No. 10/183,144B2 disclosing a ultraviolet sterilizing drainage catheter comprising at least one optical fiber disposed on the inner wall facing the longitudinal interior space and outside of the interior space not parallel to an axis of the lumen and adapted to receive the UV light from the UV illumination coupler and to emit UV light into the lumen along at least a portion of a length of the lumen.

There are two routes of infection, i.e., extraluminal (infection from outer surface of catheter) and intraluminal (infection from inner surface of catheter). Although UV based clip on devices are known in the prior-art, in view of the prior art solutions disclosed hitherto, there exists a dire need an improved device that prevents both extraluminal and intraluminal routes of infections in a urinary catheter.

### SUMMARY OF THE INVENTION

The following disclosure presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

An object of the present invention is to provide a catheter that prevents the development of catheter associated urinary tract infections, both extraluminal and intraluminal.

Another object of the present invention is to provide a device having means for inline disinfection of a urinary catheter.

Yet another object of the present invention is to provide an add-on device for a urinary catheter that will not change the existing catheter system and procedure.

In an aspect of the invention is disclosed an add-on device for connecting between urinary catheter drainage tube junctions comprising a clip-on device comprising a transducer producing surface acoustic wave (SAW) for preventing extraluminal route of infection of the tube and a source of electromagnetic radiation for preventing the intraluminal route of infection in the tube, wherein the clip on device is attached on to a coupler connecting the junctions of the catheter.

In another aspect of the invention is disclosed a urinary catheter comprising an add-on device disclosed in the previous aspect.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE ACCOMPANYING DRAWINGS

The above and other aspects, features, and advantages of certain exemplary embodiments of the present invention will be more apparent from the following description taken in conjunction with the accompanying drawings in which:

FIG. 1 illustrates a clip-on device to prevent catheter associated urinary tract infections, in accordance with the present invention.

FIGS. 2(a)-(e) illustrate the different perspective view of the clip-on device and the coupler in accordance with the present invention.

FIG. 3 illustrates a coupler attached between the catheter and the collection bag, in accordance with an embodiment of the present invention.

FIGS. 4(a)-(b) illustrate the assembly of the add-on device along with the catheter system in accordance with the present invention.

FIG. 5 illustrates that the optical fibre is embedded vertically in the catheter wall, in accordance with an embodiment of the present invention.

FIG. 6 illustrates the inline optical fibre, in accordance with an embodiment of the present invention.

FIG. 7 illustrates the inline optical fibre mesh, in accordance with an embodiment of the present invention.

FIG. 8 illustrates the inline horizontal optic array ring, in accordance with an embodiment of the present invention.

FIG. 9 illustrates the inline vertical optic array ring, in accordance with an embodiment of the present invention.

FIG. 10 illustrates the TiO₂ cap+external light source, in accordance with an embodiment of the present invention.

FIG. 11 illustrates a catheter and/or coupler whose inner walls are coated with TiO₂, in accordance with an embodiment of the present invention.

FIG. 12 illustrates the embedded piezoelectric transducer inside catheter and/or coupler, in accordance with an embodiment of the present invention.

FIG. 13 illustrates light transmissive coupler with clip on consisting of LEDs outside the coupler, in accordance with an embodiment of the present invention.

FIG. 14 illustrates coupler embedded with inline LEDs, in accordance with an embodiment of the present invention.

FIGS. 15(a)-(b) illustrate the add-on device along with the strap in accordance with the present invention.

FIG. 16 illustrates the experimental set up used for proof of concept of the device according to the present invention.

FIG. 17 (a)-(b) illustrate the device in accordance with the present being used in the body of the patient.

FIGS. 18 (a)-(b) illustrates a clip-on and a coupler device with the reflective materials for reflecting the electromagnetic radiation to the inside of a coupler and the shield materials for preventing leakage of the electromagnetic radiation.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE PRESENT INVENTION

The following description with reference to the accompanying drawings is provided to assist in a comprehensive understanding of exemplary embodiments of the invention. It includes various specific details to assist in that understanding but these are to be regarded as merely exemplary.

Accordingly, the persons skilled in the art will recognize that various changes and modifications of the embodiments described herein can be made without departing from the scope of the invention. In addition, descriptions of well-known functions and constructions are omitted for clarity and conciseness.

The terms and words used in the following description are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by their equivalents.

It is to be understood that the singular forms “a”, “an,” and “the” include plural referents unless the context clearly dictates otherwise.

By the term “substantially” wherever used or will be used later it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

In the present invention, “Coupler” (3) refers to a joint part between urinary catheter and urine collection bag. In following in some embodiment of the invention is disclosed a modified coupler, for UV light through transmissive glass. In another embodiment, the coupler design can be changed in case of optics fibres are used as source of light. As appreciated by the skilled person, based on the design of the coupler, the different tube and connection port will be suitably required.

In the present invention, “Inline” refers to single/multiple optical fibres inside the tube or connection port.

In the present invention, “Mesh” refers to an optical fibre meshes inside the tube or connection port.

In the present invention, “Cap” refers to a small device which is located inside the tube or connection port.

The subject invention lies in providing a device to prevent catheter associated urinary tract infections. Since UV based clip on devices are known in the prior-art, the present invention discloses an alternative solution for reducing catheter-associated urinary tract infections by disinfecting the microorganisms present on the inside and the outside of the catheter with minimal power requirements. The clip-on device can be attached at the connection port or on the coupler or urine collection bag tube or the urinary catheter. It irradiates UV/other spectrum light from the outside of catheter and kill bacteria inside and outside of the catheter and may employ photo-catalysts for assisting with effective disinfection at low power consumption.

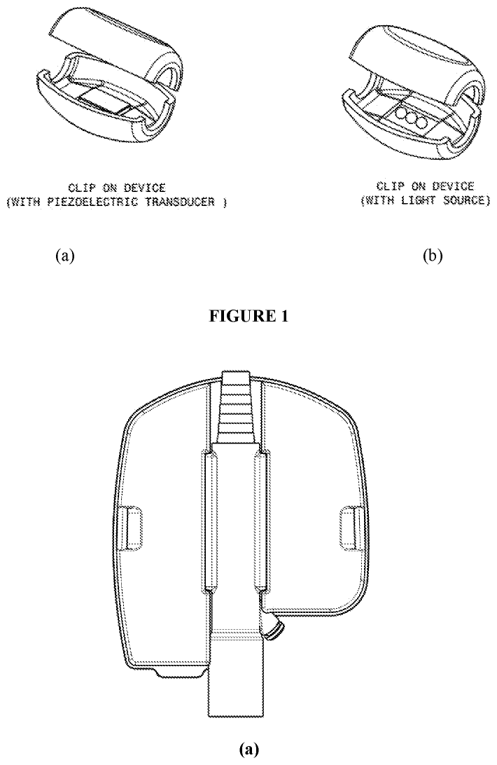

The “clip-on” device (4) of FIG. 1 has the energy source of vibration or light as well as electronic circuit components. FIG. 1(a) illustrates the clip-on device that employs a piezoelectric transducer to achieve the same while FIG. 1(b) illustrates the clip-on device that employs UV/visible light/other light spectrum. The clip-on may have at least one vibration transducer which produces the nano-vibration or surface acoustic waves or ultrasonic wave or Rayleigh wave, love wave or any other waves or combination of two or more type of waves. The said transducer produces vibration continuously or it can produce intermittently. The frequency of vibration ranges from 1 KHz to 500 MHz.

The clip-on device employing UV light or piezoelectric transducer or magnetostrictive transducer or electromagnetic induction transducer consists of a combination of the follow features:
A connector element (1a, 1b) with optical fibres, which allows UV access to the catheter for antimicrobial activity along with a clip-on device, placed over the connector for disseminating UV light. According to an embodiment of the invention, the coupler may be designed to perform the function of a connector while in other designs there may be a separate connector to connect the coupler with the clip-on device.
A light transmissive coupler, which allows UV access to the catheter for antimicrobial activity along with a clip-on device, placed over the connector for disseminating UV light.
A piezoelectric element for dislodgement of biofilm formation in urinary catheter.
The combination of UV light for antimicrobial activity and photo catalyst material for increased effectiveness.
The combination of UV light for antimicrobial activity and piezoelectric transducer for preventing biofilm formation.
The combination of UV light for antimicrobial activity and photo-catalyst material along with piezoelectric transducer for preventing biofilm formation.
Combination of UV light and magnetostrictive transducer for preventing CAUTI or magnetostrictive transducer alone for preventing CAUTI.

Significantly, the usage of both UV light and vibration technologies in combination will have greater impact on reduction of infection rates. The vibration will act on dislodging the biofilm while UV will kill the bacteria. The non-obviousness lies in the various ways of exposing urine flow to light source (5). The embodiments with inline light source (UV LEDs) are different than the prior art. These in combination with vibration will increase the effectiveness significantly.

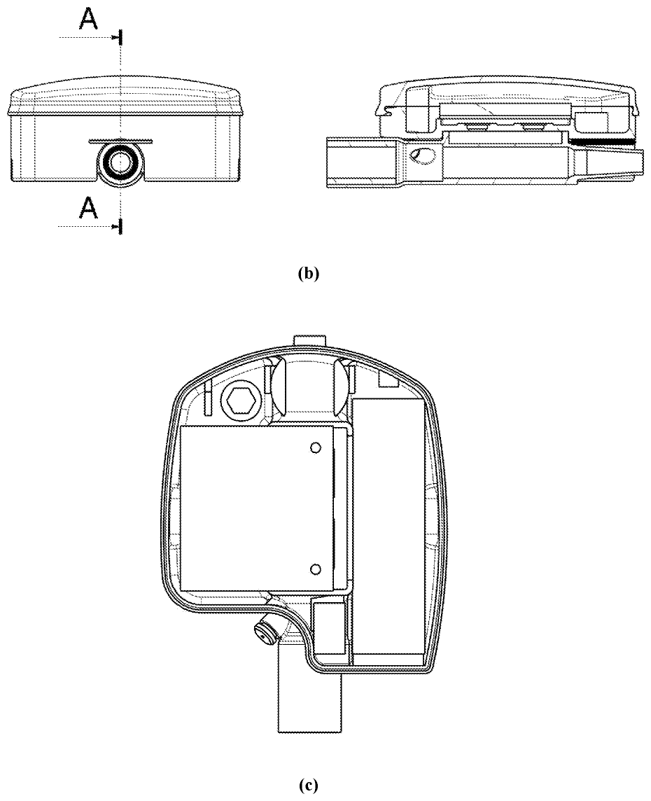

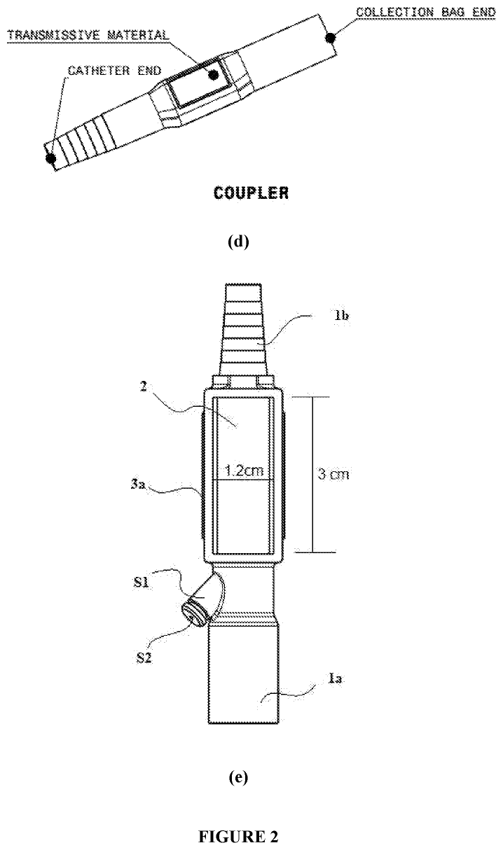

In FIG. 2(a) the coupler mechanically coupled with the clip-on device (4) is illustrated. This shows the external view of the device. The coupler is operably coupled to the clip-on device (4). The clip-on device (4) comprises grooves and/or protrusions into which the coupler fits. The grooves and/or protrusions may be surrounded with supports to hold the coupler (3) in position. FIG. 2 (c) provides the internal view of the clip-on device (4) coupled with the coupler in which the figure shows the clip-on device comprising the electronic components for the working of the device. FIG. 2(b) provides the sectional view of the clip-on device according to an embodiment of the present invention. The coupler according to an embodiment of the invention is shown in FIGS. 2(d) and 2(e). The tapering end is the end that connects with the catheter while the thicker end connects at the collection bag. The middle portion of the coupler (3) is made of a transmissive material. The coupler is made of light transmissive or transparent material. Light here implies UV/visible light/other light spectrum. Complete coupler body may or may not be made of same material. The coupler (3) is made of at least one part of transmissive or transparent material adapted to allow maximum transmission of the electromagnetic radiation. The transmissive or transparent part of coupler (3) is made of fused silica glass, quartz glass or any other materials having at least more than 50% transmittivity for the UV radiation.

Reference is made to the embodiments of the coupler disclosed in FIG. 2 (d) horizontally and FIG. 2 (e) vertically. The figures depict that the coupler includes a window (2) of transmissive material for irradiating the electromagnetic radiation. The window (2) is having a length of approximately 3 cm and a width of 1 to 1.5 cm. Bacteria usually propagate in urinary catheter at the rate of 2.5 cm per hour. So, the minimum length of the window (2) for optimum exposure to the bacteria to disinfect using electromagnetic radiation is 3 cm. To produce the desired effect of disinfection the length of the window (2) should not be less than 3 cm, when the device irradiates electromagnetic radiation 5-10 minutes every hour. The optimum size of the window (2) also depends on the radiation period and frequency of electromagnetic radiation by the device. Hence, if the device irradiated longer ore more frequently, then it is possible to modify size of the window (2).

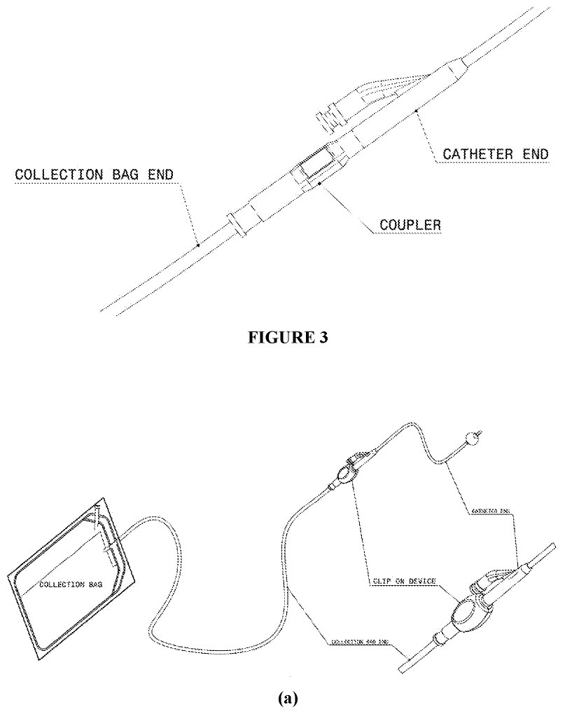

FIG. 3 illustrates a coupler (3) which is attached as an intermediary between the catheter and the collection bag. The coupler material is one which allows maximum UV transmission.

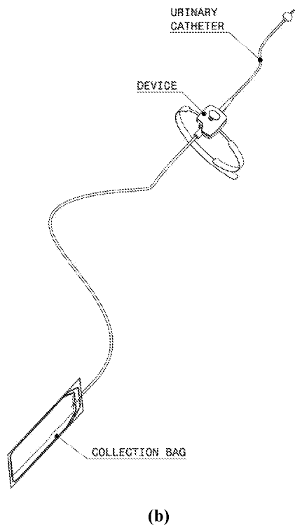

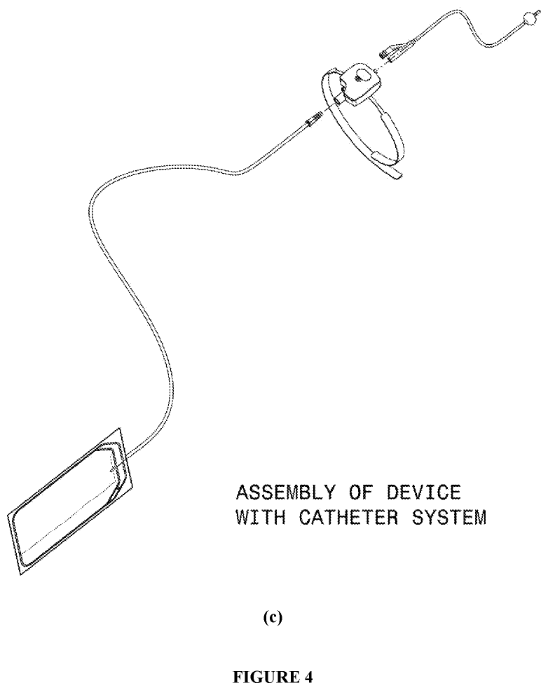

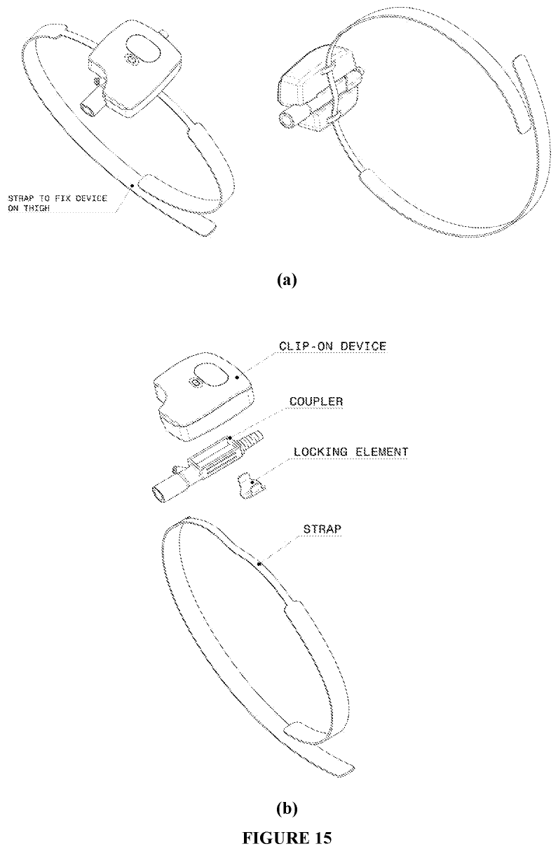

FIG. 4 (a) illustrates Clip on device (4) attached onto coupler which is connected between the catheter and the bag. FIG. 4(b) shows the assembly of the device with the catheter system along with a strap (9) to hold the add-on device to the thigh of the patient, according to an embodiment of the invention. The coupler is attached in between catheter and urine collection bag. Clip on device (4) which fits on top of the coupler consists of energy source such as light and/or vibration and/or magnetic field which kills the bacteria in coupler (3) as well as inhibits the movement of bacteria from urine bag to the catheter tip. The coupler (3) and clip-on (4) are separable. The coupler may be for one-time use (disposable), but the clip-on device (4) which includes light sources and battery etc. can be used multiple times. The exploded view of the assembly of the device with the catheter system is seen in FIG. 4(c). FIG. 15(a) shows the different view of the add-on device along with the strap (9). In FIG. 15(b), the different parts of the embodiment in FIG. 15(a) is shown in an exploded view.

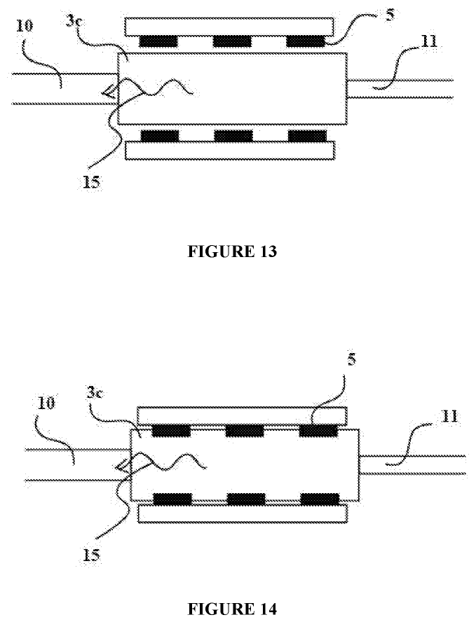

FIG. 13 illustrated coupler (3) made of light transmissive material. The clip on device consists of UV/visible light source (5) which fits onto the coupler (3). Coupler may or may not be coated with photo catalyst material. Bacterial growth is inhibited by exposure to UV light.

FIG. 14 illustrates coupler (3) embedded with inline UV/visible light source (5). Light source is not in direct contact and juts out in the lumen of the coupler (3). Coupler may or may not be coated by photo catalyst material.

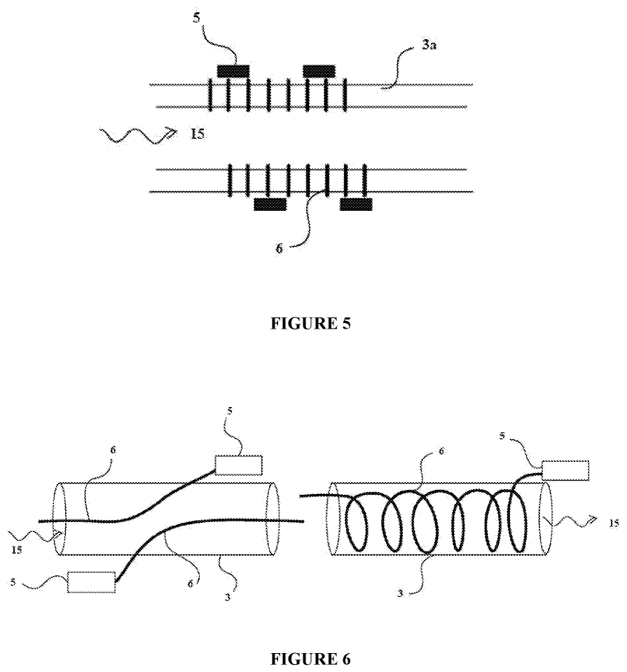

FIG. 5 illustrates that the optical fibre (6) is embedded vertically in the catheter wall. This inhibiting the growth and/or kills the bacteria on the inner wall of the catheter surface by exposing it to UV radiation. The fibre optic cables (6) are inserted into the lumen wall (not inserted into the urine stream). UVC is transmitted through the fibre optic cables into the urine stream as the catheter material used in is highly transmissive. Significantly, the optical fibres just touch the inner wall and do NOT jut out.

FIG. 6 illustrates a coupler (3) with inline optical fibre (7). The coupler (3) has optical fibres which make light (UV/visible light) passes into the inside. The optical fibres are exposed into the urine path (15) directly. Light is irradiated from outside into inside of coupler via optical fibre, in order to disinfect bacteria. The TiO₂ coats on the optical fibres or inside surface of tube. This kills the bacteria with only UV, or combination of TiO₂ and light (UV or visible light). It is possible to use a straight optical fibre, curled optical fibre, waved optical fibre and so on and use single optical fibre or multiple optical fibres.

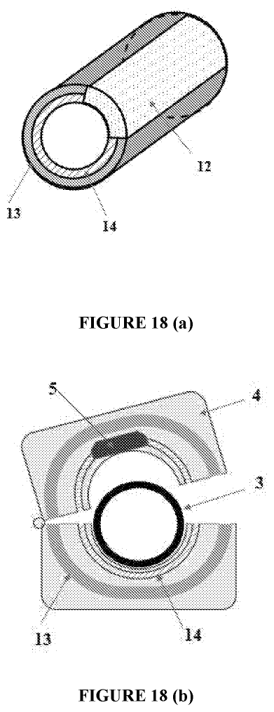

FIG. 18 (a) and FIG. 18 (b) illustrate the coupler (3) and/or the clip-on (4) have reflective material to reflect the electromagnetic radiation within the urine channel, in order to get maximum effectiveness of electromagnetic radiation. Further, the coupler and/or the clip-on have a shielding material (13) preventing leakage of electromagnetic radiation (such as and not limited to UV-C) from the device. The preventing leakage may be implemented by the mechanism of connection between the coupler (3) and the clip-on device (4). It is necessary to prevent the leakage of electromagnetic radiation from the device because some kind of electromagnetic radiation is harmful to humans, thereby ensuring the safety aspect of the device. The clip-on (4) and coupler device (3) may operate coupled to prevent the leakage of electromagnetic radiation out of the device. The clip-on and the coupler includes with the reflective materials (12) for reflecting the electromagnetic radiation to the inside of a coupler and the shield materials (13) for preventing leakage of the electromagnetic radiation.

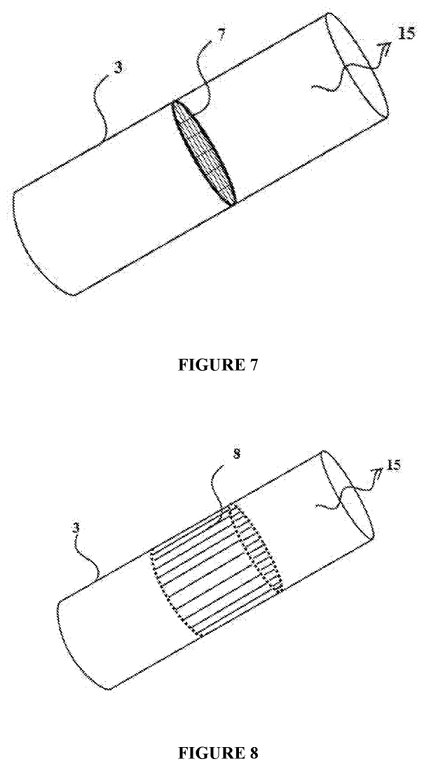

FIG. 7 illustrates the inline optical fibre mesh (7). It consists of a coupler which has a ring or mesh of side emitting optical fibres. These fibres emit the lights of possible combinations mentioned above. Coupler or optical fibre may or may not be coated with photo catalyst material. Coupler or connector design will be different to accommodate the optic fibres be it in the form of ring or mesh. One possible design the coupler may be made of transmissive material (12) with LEDs (5) arranged linearly for disinfection of the bacteria. In an alternate possible design, the coupler may include fibre optic ring or mesh for disinfection. Both the designs may or may not include the photocatalyst coating (16).

FIG. 8 illustrates a coupler attached as an intermediary between the catheter and the collection bag. The coupler is made up of fibre optic array ring (8). The fibre optic array ring is placed horizontally. Coupler (3) or optical fibre may or may not be coated with photo catalyst material.

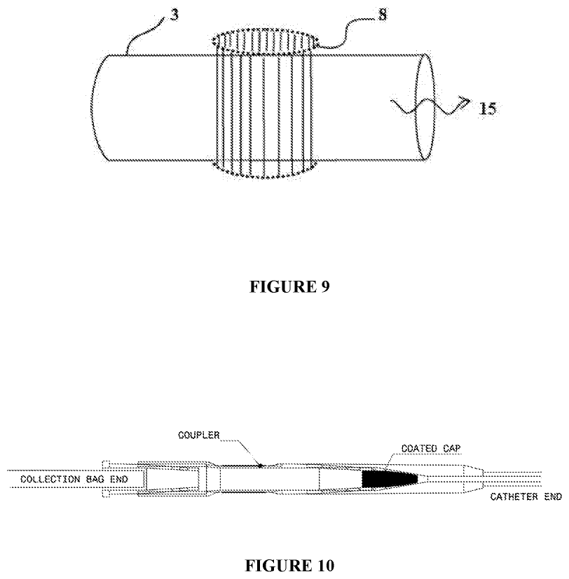

FIG. 9 illustrates a coupler attached as an intermediary between the catheter and the collection bag. The coupler is made up of fibre optic array ring. The fibre optic array ring is placed vertically. Coupler or optical fibre may or may not be coated with photo catalyst material.

FIG. 10 illustrates the photo catalyst cap with external light source. The Cap is coated with photo catalyst material such as TiO2 and is located inside the tube or connection port. External light source (5) such as UV/visible light can be provided by using clip-on device or environmental lighting.

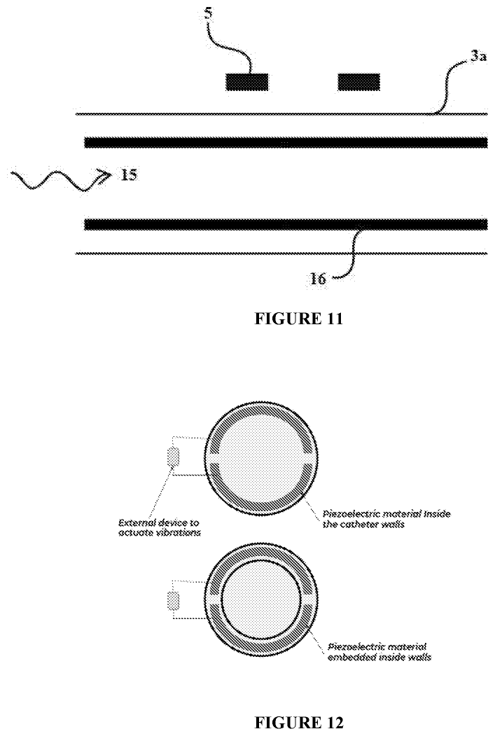

FIG. 11 illustrates a catheter whose inner walls are coated with photo catalyst such as TiO₂. Similar coating can also be applied on the inner walls of the coupler as well. The clip-on device or energy source (5) can be fitted anywhere at exposed catheter tube.

FIG. 12 illustrates a catheter with embedded piezoelectric transducer. Piezoelectric transducer may be located in inner surface of the catheter or outside the catheter or inside catheter walls. The piezoelectric material is embedded with the catheter walls and the external device actuates the vibrations. Similar arrangement is possible with coupler as well.

The vibration transducer touches surface of the coupler or catheter or drainage tube or all of them. The vibration transducer may be embedded with piezoelectric material or vibration motor. Piezoelectric material will be fixed or embedded with the clip-on device as clip-on device is reusable. And piezoelectric material can contact with outer surface of the coupler or catheter surface or drainage tube surface or all of them. In an embodiment the shape of piezoelectric material is circular or disc, cylindrical, a half cylindrical or any other form.

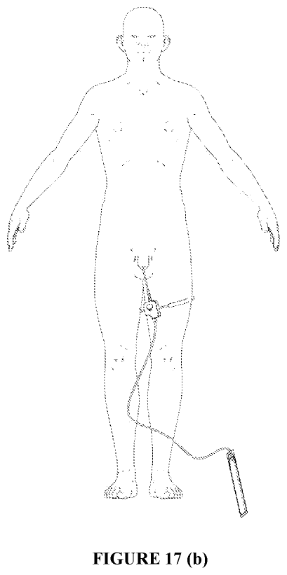

In the present invention, disinfection is achieved by UV light (mainly UV-C), by photocatalyst (TiO₂) with UV light (mainly UV-A); and by improved photocatalyst (TiO₂) with visible light. Irradiating light to target area including bacteria is done from outside of the urinary catheter or urine collection bag. The device may be attached on the urinary catheter outside of body, attached on tube of the urine collection bag, attached on junction point between catheter and collection bag. It can also be connected between urinary catheter and urine collection bag like a joint part. The tube is transparent for UV light (UV-A or UV-C), In particular, UV-C for killing bacteria by UV irradiation and UV-A for killing bacteria by photocatalyst (TiO₂). The tube has a side emitting optical fibre coating with photocatalyst (TiO₂), in order to sterilize bacteria around connection port between catheter and the tube. The device can switch UV/visible light source (5) ON continuously or intermittently for saving energy. For example, it may be on for 10 min and is off for 50 min. Significantly, it turns on the LEDs until it kills most of the bacteria causing UTI. There are several variations of the arrangement of UV LEDs such as linear arrangement of FIG. 13 or 14. The UV irradiation area may be covered with reflection material for increasing UV intensity in the catheter. FIGS. 17 (a) and (b) show the device disclosed herein being used in the body of the patient. FIG. 17 (a) shows the lateral view of the patient's body while FIG. 17 (b) shows the top view of the patient's body.

### EXPERIMENTAL DATA

### Experiment 1-UVC Kills the Bacteria

UV-C Radiation source: LED, 253.4 nm, intensity-4 w, distance-10 cm)
Experiment done: 29/01/2019, 4:30 pm
(Incubated in Biological Oxygen Demand, (BOD) at 37 degree centigrade for >16 hrs)
Observation: 30/01/2019, 10:15 am
The Results are:

| | | | Colony | Colony | |
|---|---|---|---|---|---|
| | Exposure | Colony | count (2) | count (2) | |
| Pathogen | time | count (1) | (x.1) | (x.1/10) | Remarks |
| E-coli | 5 min | 11 | 6 | 1 | |
| E-coli | 10 min | 1 | 2 | 3 | |
| E-coli | 15 min | 4 | 1 | 1 | |
| E-coli | 20 min | 5 | 1 | 1 | |
| E-coli | 0 (+ve | Full | Full | Full | |
| | Control) | growth | growth | growth | |
| E-coli | 0 (−ve | 0 | 1 | 1 | |
| | control) | | | | |

### Experiment 2—UVC Kills the Bacteria

Experiment: 31/01/2019, 3:30 pm
(Incubated in BOD at 37 degree centigrade for >16 hrs)
Observation: 01/02/2019, 10:40 am
The results are:

| | | | | |
|---|---|---|---|---|
| | | 10\hat{ }3 cfu/ml | 10\hat{ }5 cfu/ml | |
| | Exposure | Or 10\hat{ }2 cfu/100 | Or 10\hat{ }4 cfu/100 | |
| Pathogen | time | micro-ml | micro-ml | Remarks |

| | | | |
|---|---|---|---|
| E-coli | 5 min | 1 | 0 |
| E-coli | 10 min | 5 | 0 |
| E-coli | +ve Control | Full growth | Full growth |
| E-coli | −ve Control | Contamination | Contamination |
| | | at 10 places | at 10 places |

* S stands for “Sample”
** ES stands for “End of experiment -Sample”
*** MALDI-TOF mass spectrometry technology is used to identify the pathogen in samples.
**** P. Rettgari means Providencia Rettgari

### Experiment 3-Determine the Effectiveness of Bacterial Disinfection Through UVC LED

Led Specifications:
Wavelength—285 nm
Intensity—2 w
Voltage—7v
Current—250 mA

The results are:

Data Collection

| | | Energy | Exposure | Start | Result | |
|---|---|---|---|---|---|---|
| Sl. | Uro- | Source | Duration | Date and | Date and | Result/ |
| No. | Pathogen | (Wavelength) | (Min) | time | time | Comments |
| 1 | E-Coli | 285 nm | 5 min | 12 Jul. 2019 | 13 Jul. 2019 | Disinfection - |
| | | | | 5 pm | 11 am | 100% |
| 2 | E- Coli | 285 nm | 10 min | 12 Jul. 2019 | 13 Jul. 2019 | Disinfection - |
| | | | | 5 pm | 11 am | 100% |
| 3 | E- Coli | 285 nm | 15 min | 12 Jul. 2019 | 13 Jul. 2019 | Disinfection - |
| | | | | 5 pm | 11 am | 100% |

This LED is effective for the current embodiment. So this LED is chosen as final for further prototyping of the device disclosed herein.

Establishment of Infection Routes

Experiment 1-6: To know the bacterial movement/routes causing infection (Using Agar coated drainage tube).

Equipment: Saline bottle, collection bag, IV set, Biosafety cabinet, Autoclave

Medium: E-coli culture, Normal Human Urine, Agar

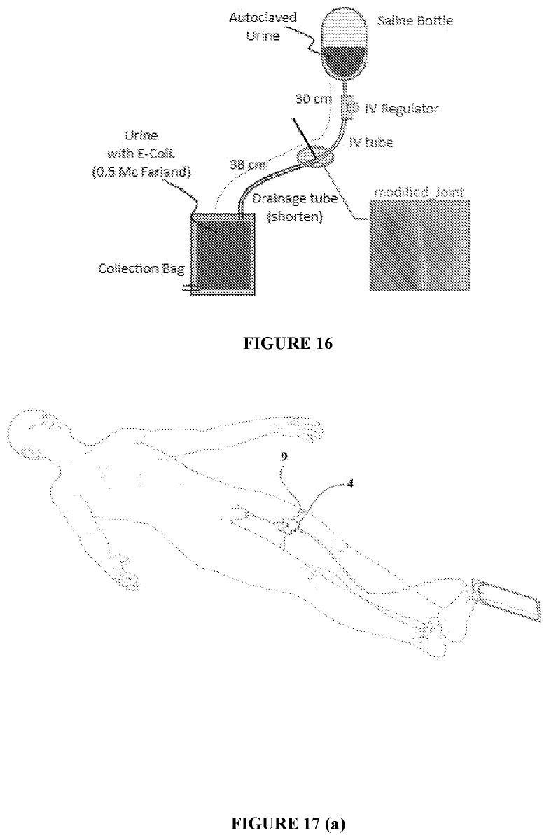

FIG. 16 shows the experimental setup used for validating the present invention. The details of the set up are provided below:
Saline bottle mimicking bladder, saline is replaced with autoclaved normal urine
Agar coated distal end drainage tube of collection bag is chopped to make it shorten and connected in IV tube
IV set, distal end is inserted into saline bottle filled with autoclaved normal urine
Autoclaved Urine (200 ml) is put in collection bag
E-coli is mixed in urine presented in collection bag (200 ml, 1.8×10\hat{ }8 concentration or 0.5 McFarland)

### Working:

Autoclaved normal urine is dropped in form of drop from the saline bottle using IV regulator
Drop of urine passes through IV line, drainage tube and then collected into collection bag
Flow rate of urine=approx. 1 drop/20 Sec

### Other Procurement:

Normal urine was collected in sterile container from a single person at 9:30 am (approx. 700 ml)
Urine was given for autoclave (at AIIMS) at 10:00 am
At 2:00 pm urine was autoclaved and it was ready for experiment
At 4:00 pm experiment was started
Specification—length used, urine etc.,
   IV tube=12 inches=30 cm
   Drainage tube=18 inches=45 cm
   Height of the bottle=15.5 inches
   Urine loaded in bottle=500 ml
   Urine loaded in bag (with culture)=200 ml

Result:

Samples were plated on petri-dish and incubated for more than 16 hrs at 37 degree centigrade, to see the bacterial growth.

Data Collection Sheet

| | | | | | | Comments | |
|---|---|---|---|---|---|---|---|
| | | | | | | Bacteria | |
| Sample | Sampling point/ | Sampling Time & Date | | Results Time & Date | | Found = Yes | Remarks/ |
| No. | Place | Time | Date | Time | Date | Not found = No | Validation*** |
| 00 | Control | 4:30 pm | 09/07 | 10:00 am | 10/07 | No | |
| S1 | Bottle urine | 5:00 pm | 09/07 | 10 am | 10/07 | No | |
| | (Autoclaved) | | | | | | |
| S2 | Bag (Urine with | 5:00 pm | 09/07 | 10 am | 10/07 | Yes | E-coli |
| | culture) | | | | | | |
| S3 | From bottle | 10:15 am | 10/07 | 10 am | 11/07 | Yes | P. Rettgari |
| | (Bladder) | | | | | | |
| S4 | From bottle | 01:20 pm | 10/07 | 10 am | 11/07 | Yes | P. Rettgari |
| | (Bladder) | | | | | | |
| S5 | From bottle | 10:22 am | 11/07 | 12 pm | 12/07 | Yes | P. Rettgari |
| | (Bladder) | | | | | | |
| S6 | From bottle | 2:15 pm | 11/07 | 12 pm | 12/07 | Yes | P. Rettgari |
| | (Bladder) | | | | | | |
| S7 | From bottle | 5:25 pm | 11/07 | 12 pm | 12/07 | Yes | P. Rettgari |
| | (Bladder) | | | | | | |
| S8 | From bottle | 3:25 pm | 12/07 | 10 am | 13/07 | Yes | P. Rettgari |
| | (Bladder) | | | | | | |

End of Experiment (Swabbing Method)

| | | | | | | | |
|---|---|---|---|---|---|---|---|
| ES1 | From point 1 bag | 9:20 am | 13/07 | 10:22 am | 14/07 | Yes | P. Rettgari |
| ES2 | From point 2 | 9:50 am | 13/07 | 10:22 am | 14/07 | Yes | P. Rettgari |
| | Drainage tube | | | | | | |
| ES3 | From point 3 | 9:50 am | 13/07 | 10:22 am | 14/07 | Yes | P. Rettgari |
| | Drainage tube | | | | | | |
| ES4 | From point 4 | 9:50 am | 13/07 | 10:22 am | 14/07 | Yes | P. Rettgari |
| | Drainage tube | | | | | | |
| ES5 | From point 5 | 9:50 am | 13/07 | 10:22 am | 14/07 | Yes | P. Rettgari |
| | Drainage tube | | | | | | |
| ES6 | From point 6 | 9:50 am | 13/07 | 10:22 am | 14/07 | Yes | P. Rettgari |
| | Drainage tube | | | | | | |
| ES7 | From point 7 | 9:50 am | 13/07 | 10:22 am | 14/07 | Yes | P. Rettgari |
| | Drainage tube | | | | | | |
| ES8 | From point 8 | 9:50 am | 13/07 | 10:22 am | 14/07 | Yes | P. Rettgari |
| | Bottle | | | | | | |

* S stands for “Sample”
** ES stands for “End of experiment -Sample”
***MALDI-TOF mass spectrometry technology is used to identify the pathogen in samples.
**** P. Rettgari means Providencia Rettgari

### Conclusion:

Bacteria are presented everywhere
E-coli Bacteria are absence
Experiment Duration: 88:50 Hrs. (9 Jul. 2019, 4:30 pm-13 Jul. 2019, 9:20 am)

| Exp. No. | Medium (E-coli with) | Tube Length | Duration | | Bacteria Status |
|---|---|---|---|---|---|
| Experiment 1 | Normal Urine | Full Length | 65 | hrs | No Bacteria found |
| Experiment 2 | Normal Urine | Shorten length | 62.5 | hrs | Bacteria found everywhere |
| Experiment 3* | Autoclaved Urine | Shorten length | 64.5 | hrs | Bacteria found at sample 2, 9, 10, 11, 12 |
| Experiment 4* | Autoclaved Urine | Shorten length | 63 | hrs | Bacteria found at sample 2 (Bag) |
| Experiment 5** | Autoclaved Urine | Shorten length | 55 | hrs | Bacteria found at sample 2, 9, 10, 11, 12, 16 |
| Experiment 6** | Autoclaved Urine | Shorten length | 88.5 | hrs | Bacteria found at sample 2, 9, 10, 11, 12, 13, 14, 15, 16 |

*Bacteria travelled = 45 cm
**AGAR coated drainage tube and bacteria travelled = 75 cm

For extraluminal route of infection, the following have been observed:
Surface acoustic wave (SAW) using piezoelectric, motor, or magnetic is being used to dislodge the biofilm on the surface of the catheter and to prevent the adherence of bacteria with catheter surface.
Currently we are using piezoelectric transducer to generate the Surface acoustic wave.

For intraluminal route of infection, the following have been observed:
UVC is able to disinfect the uropathogen causing infection (CAUTI).
Intensity required to disinfect the uropathogen is optimised.
Exposure time of UVC to uropathogen is optimised.

Some of the important and noteworthy advantages of the present invention, considered to be noteworthy are mentioned herein below:
An add-on device with existing urinary catheter which can be easily acceptable. It can be easily integrated with current usage scenario in medical facilities.
High UV exposure in and out of the catheter tube with enhanced effect in presence of photo catalyst such as TiO₂.
Combined use of UV/visible light exposure and vibration to enhance the effectiveness of UTI prevention.
Inline exposure of urine flow to UV/visible light source to increase efficiency.
Use of alternating magnetic field to inhibit growth of biofilms and kill bacteria.
Invention technology can be extended to be used for other applications where catheters are used.

Although a way to address catheter-induced urinary tract infections in order to reduce patient morbidity has been described in language specific to structural features and process steps, it is to be understood that the embodiments disclosed in the above section are not necessarily limited to the specific features or components or devices or methods described. Rather, the specific features are disclosed as examples of implementations for a device to prevent catheter associated urinary tract infections.

The herein disclosed solution is designed for preventing both intraluminal and extraluminal routes of infections. It is a small an add-on device to be connected between the catheter drainage tube junctions. It utilizes the principle of UV irradiation in combination with photocatalyst (16) to kill the bacteria travelling in the catheter via intraluminal route. The device also releases low-frequency ultrasonic waves which prevents the adhesion of the bacteria on the external surface of the catheter. Thus, it prevents the CAUTI by not allowing bacteria to enter the urinary tract by any of the routes.

### LIST OF REFERENCE NUMERALS AND THE CORRESPONDING FEATURES

1a: connector
1b: connector
2: window
3: coupler
3a: coupler wall
3c: coupler lumen
4: clip-on device
5: source of electromagnetic radiation or LED
6: Optic fibre
7: inline optical fibre mesh
8: fibre optic array ring
9: strap
10: bag end
11: catheter end
12: Transmissive material
13: shield material
14: reflective material
15: urine flow
16: photocatalyst coating
S1 and S2: the means provided in the coupler to collect the urine sample

1. An add-on device for connecting between a urinary catheter and a drainage tube of a urine collection bag comprising:
a clip-on device (4) comprising a body including:
a source of electromagnetic radiation; and
a clip portion; and
a tubular coupler (3) having a window (2) adapted for transmitting electromagnetic radiation;
wherein the clip-on device (4) is attached to the tubular coupler (3) by the clip portion mating with the tubular coupler.

2.-4. (canceled)

5. The add-on device of claim 1, wherein the window (2) is made of transmissive or transparent material.

6. The add-on device of claim 5, wherein the window is made of fused silica glass or quartz glass or other materials having at least 50% transmittivity for UV radiation.

7. (canceled)

8. The add-on device of claim 1, wherein the source of electromagnetic radiation (5) is adapted to fit onto the tubular coupler (3) such that electromagnetic radiation emitted from the source of electromagnetic radiation (5) disinfects bacteria inside the tubular coupler (3) and/or at a junction of the tubular coupler (3) and h catheter, and/or at junction of the tubular coupler (3) and the drainage tube.

9. The add-on device of claim 1, wherein the source of electromagnetic (5) radiation emits electromagnetic radiation intermittently.

10. The add-on device of claim 1, wherein the source of electromagnetic radiation (5) is UV light source (5).

11. (canceled)

12. The add-on device of claim 1, wherein the tubular coupler (3) comprises an optic fibre configured to transmit electromagnetic radiation from the source of electromagnetic radiation (5).

13. The add-on device of claim 12, wherein the optical fibre (6) is a straight optical fibre, a curled optical fibre, a waved optical fibre, or a combination thereof and is single optical fibre or multiple optical fibre; and
wherein a fibre optic array ring (8) is placed in the coupler (3) vertically or horizontally and a mesh (7) of side emitting optical fibres.

14. The add-on device of claim 12, wherein the optical fibre (6) and/or the tubular coupler (3) is coated with TiO₂ or other photo catalyst material (16) that assists with disinfection.

15. The add-on device of claim 1, wherein the tubular coupler (3) or/and the clip-on (4) device comprises a reflective material (14) or coating of reflective material to reflect the electromagnetic radiation within a urine channel.

16. The add-on device of claim 1, wherein the tubular coupler (3) or/and the clip-on device (4) comprise a shield material (13) adapted to prevent leakage of electromagnetic radiation.

17. The add-on device of claim 1, wherein the tubular coupler (3) and the clip-on device (4) are operably coupled to prevent the leakage of electromagnetic radiation out of the device.

18. The add-on device of claim 1, wherein the clip-on device (4) comprises at least one vibration transducer producing nano-vibration or surface acoustic waves (SAW) or ultrasonic wave or Rayleigh wave or love wave or any other waves or combination of two or more types of waves.

19.-38. (canceled)
